(19) United States
(12) Patent Application Publication
(10) Pub. No.: US 2025/0387456 A1
(43) Pub. Date: Dec. 25, 2025

(54) TARGETING NANOENZYME FOR MITIGATING CHEMOTHERAPY-INDUCED CARDIOTOXICITY, PREPARATION METHOD

(71) Applicants: Junyue Xing, Beijing (CN); Hao Tang, Beijing (CN)

(72) Inventors: Junyue Xing, Beijing (CN); Hao Tang, Beijing (CN); Wei Jiang, Henan (CN); Ying Liu, Henan (CN); Yanan Yu, Henan (CN); Yingying Wang, Henan (CN); Xiaohan Ma, Henan (CN); Lu Chen, Henan (CN); Weining Yuan, Henan (CN)

(73) Assignees: Junyue Xing, Beijing (CN); Hao Tang, Beijing (CN)

(21) Appl. No.: 19/231,593

(22) Filed: Jun. 9, 2025

(30) Foreign Application Priority Data
Jun. 21, 2024 (CN) 202410816578.8
Sep. 5, 2024 (CN) 202411243334.1

(51) Int. Cl. A61K 38/43; A61P 9/00; C12N 9/02; C12N 9/08

(52) U.S. Cl. CPC A61K 38/43; A61P 9/00; C12N 9/0065; C12N 9/0089; C12Y 111/01006; C12Y 115/01001

(57) ABSTRACT

Disclosed is a method for preparing Au-Ru nanoenzymes, with steps as: 1) dissolving HAuCl₄ and ruthenium metal salt in solvent to obtain metal salt solution; 2) adding NAC and TA to the metal salt solution, mixing them uniformly to obtain mixture; 3) adding NaBH₄ aqueous solution to the mixture, stirring to react at room temperature, then continuing to stir to react at 50° C.˜70° C.; after the reaction is finished, performing centrifuging, and the precipitate is lyophilized to obtain Au—Ru nanoenzymes. The present invention further discloses a method for preparing ATBMzyme nanoenzymes, specifically: adding EDC and NHS to Au—Ru nanoenzymes solution, stirring to react, then adding brain natriuretic peptide to the reaction system, continuing to stir to react; after the reaction is finished, performing centrifuging, and the precipitate is lyophilized to obtain ATBMzyme nanoenzymes.

### CROSS-REFERENCE TO RELATED APPLICATION

This application claims the priority benefit of China application serial no. 202410816578.8, filed on Jun. 21, 2024 and China application serial no. 202411243334.1, filed on Sep. 5, 2024. The entirety of each of the above-mentioned patent applications is hereby incorporated by reference herein and made a part of this specification.

### BACKGROUND

### Technical Field

The present disclosure belongs to the field of biomedicine, specifically relating to a targeting nanoenzyme for mitigating chemotherapy-induced cardiotoxicity and preparation method thereof.

### Description of Related Art

Anthracycline drug doxorubicin is currently the main ingredient of anti-cancer treatment regimens used clinically. However, early-stage studies have indicated that anthracycline chemotherapy drugs might cause cardiac-related toxic side effects, including arrhythmia and cardiomyopathy, which have become limiting factors restricting their clinical application. How to relieve chemotherapy-induced cardiotoxicity and explore effective intervention measures is urgently needed.

For the cardiac-related toxic side effects caused by anthracycline drugs, there are currently many hypotheses, including changes in cell death pathways, mitochondrial dysfunction, etc. Oxidative stress is an important mechanism of anthracycline drug-induced cell death. Nanoenzymes are a class of enzyme mimics that have both the unique properties of nanomaterials and catalytic functions, with many advantages compared with nanomaterials and natural enzymes, and are widely used clinically. In particular, nanoenzymes that mimic enzymes with redox activity mainly participate in the regulation of ROS in vivo and in cells. Whether their inhibition of ROS may be used to relieve chemotherapy-induced cardiotoxicity, and how to provide their targeting to the heart requires further research and discussion.

### SUMMARY

The first aspect of the present disclosure provides a method for preparing Au—Ru nanoenzymes, including the following steps:
(1) Dissolving HAuCl₄ and ruthenium metal salt in solvent to obtain metal salt solution;
(2) Adding N-acetylcysteine (NAC) and tannic acid (TA) to the metal salt solution, mixing uniformly to obtain a mixture;
(3) Adding NaBH₄ aqueous solution to the mixture, stirring and reacting the mixture at room temperature for 0.5 h˜2 h, then continuing to stir and react the mixture at 50° C.˜70° C. for 2 h˜4 h; after the reaction is finished, collecting the precipitate by centrifuging, then the precipitate is lyophilized to obtain Au—Ru nanoenzymes.

The molar ratio of the HAuCl₄ to ruthenium metal salt is (1.5˜6):2; more preferably, the molar ratio of the HAuCl₄ to ruthenium metal salt is 3:2.

The molar ratio of N-acetylcysteine to HAuCl₄ is (1˜10):1; more preferably, the molar ratio of N-acetylcysteine to HAuCl₄ is 2.7:1.

The molar ratio of the tannic acid to HAuCl₄ is (1˜10):(1˜10); more preferably, the molar ratio of the tannic acid to HAuCl₄ is 2:5.

The molar ratio of NaBH₄ to HAuCl₄ is (1˜8):(0.15˜0.6); more preferably, the molar ratio of NaBH₄ to HAuCl₄ is 1.25:0.15.

The concentration of the NaBH₄ aqueous solution is 0.2˜0.4 mol/L. More preferably, the concentration of the NaBH₄ aqueous solution is 0.2 mol/L.

In step (3), before adding the NaBH₄ aqueous solution to the mixture, the NaBH₄ aqueous solution needs to be pre-cooled to avoid subsequent reaction proceeding too quickly. After pre-cooling treatment, the temperature of the NaBH₄ aqueous solution is 4° C.

The second aspect of the present disclosure provides Au—Ru nanoenzymes prepared by the preparation method of Au—Ru nanoenzymes according to the first aspect as described above.

The third aspect of the present disclosure provides a method for preparing ATBMzyme nanoenzymes, the method including: dissolving the Au—Ru nanoenzymes described in the second aspect in water to obtain an Au—Ru nanoenzymes solution; adding 1-ethyl-3-(3-dimethylaminopropyl)carbodiimide (EDC) and N-hydroxysuccinimide (NHS) to the Au—Ru nanoenzymes solution, stirring and reacting the mixture, and then adding brain natriuretic peptide (ANP) to the reaction system, continuing to stir and react the mixture; after the reaction is finished, collecting the precipitate by centrifuging, and the precipitate is lyophilized to obtain the ATBMzyme nanoenzymes.

The mass ratio of Au—Ru nanoenzymes to 1-Ethyl-3-(3-dimethylaminopropyl)carbodiimide is (1˜4):(1˜20); more preferably, the mass ratio of Au—Ru nanoenzymes to 1-Ethyl-3-(3-dimethylaminopropyl) carbodiimide is 2:1.

The mass ratio of Au—Ru nanoenzymes to N-hydroxysuccinimide is (5˜20):(4˜15); more preferably, the mass ratio of Au—Ru nanoenzymes to N-hydroxysuccinimide is 5:2.

The mass ratio of brain natriuretic peptide to Au—Ru nanoenzymes is (0.2˜1):(5˜50); more preferably, the mass ratio of brain natriuretic peptide to Au—Ru nanoenzymes is 1:50.

After adding 1-ethyl-3-(3-dimethylaminopropyl)carbodiimide (EDC) and N-hydroxysuccinimide (NHS) to the Au—Ru nanoenzymes solution, the mixture is stirred and reacted for 20˜90 min.

The reaction time for stirring after adding brain natriuretic peptide to the reaction system is 8˜16 h.

Application of the above Au—Ru nanoenzymes or ATBMzyme nanoenzymes in preparing drugs for preventing, relieving or/and treating cardiotoxicity caused by chemotherapy drugs.

Application of the above ATBMzyme nanoenzymes in preparing drugs for preventing, relieving and/or treating cardiotoxicity caused by chemotherapy drugs.

The present disclosure provides a drug for preventing, relieving or/and treating cardiotoxicity caused by chemotherapy drugs, wherein the drug contains Au—Ru nanoenzymes or ATBMzyme nanoenzymes.

The chemotherapy drug is at least one of antitumor antibiotics, antimetabolites, alkylating agents, antitumor hormones, and antitumor plant component drugs.

The chemotherapy drug is at least one of DOX (doxorubicin), 5-FU (5-fluorouracil), cisplatin, cyclophosphamide, tamoxifen, and paclitaxel.

The drug also contains pharmaceutically acceptable carriers/excipients.

The carrier/excipient includes (but is not limited to): diluents, excipients such as lactose, sodium chloride, glucose, urea, starch, water, etc., fillers such as starch, sucrose, etc.; binders such as monosaccharide syrup, glucose solution, starch solution, cellulose derivatives, alginates, gelatin and polyvinylpyrrolidone; wetting agents such as glycerin; disintegrants such as dry starch, sodium alginate, kelp polysaccharide powder, agar powder, calcium carbonate and sodium bicarbonate; absorption promoters quaternary ammonium compounds, sodium dodecyl sulfate, and son; surfactants such as polyoxyethylene sorbitan fatty acid esters, sodium dodecyl sulfate, glyceryl monostearate, cetyl alcohol, and so on; humectants such as glycerin, starch, and so on; adsorbent carriers such as starch, lactose, bentonite, silica gel, kaolin and soap clay, and so on; lubricants such as talc, calcium and magnesium stearate, polyethylene glycol, boric acid powder, and so on.

Compared with the related art, the present disclosure achieves the following positive and advantageous effects:
(1) The present disclosure uses NAC, TA, metal ion Au and metal ion Ru as ligands to prepare and synthesize Au—Ru nanoenzymes through the interaction between ligands to firmly bind these ligands on the supporting material formed by the action of NAC and TA. NAC may stabilize the structure between metal ion Au and metal ion Ru. TA may not only extend the in vivo circulation time of the nanoenzyme, but also has cardiac targeting effect. The Au—Ru nanoenzymes prepared by the present disclosure has good antioxidant capability, is able to remove reactive oxygen species (ROS) and reactive nitrogen species (RNS), and has good cardiac targeting, which may be used for preventing, relieving or/and treating cardiotoxicity and cardiac injury caused by chemotherapy drugs without affecting the killing power of chemotherapy drugs on tumors as well as the function of other tissues, and therefore has high safety.
(2) The present disclosure incorporates brain natriuretic peptide (ANP) onto Au—Ru nanoenzymes to further enhance their cardiac targeting function, using 1-ethyl-(3-dimethylaminopropyl)carbodiimide (EDC) and N-hydroxysuccinimide (NHS) as catalysts to increase the reaction efficiency in synthesizing ATBMzyme, resulting in ATBMzyme nanoenzymes capable of targeting the heart. This ATBMzyme nanoenzymes has good cardiac targeting properties and excellent antioxidant capability, able to remove reactive oxygen species (ROS) and reactive nitrogen species (RNS). Such ATBMzyme nanoenzymes may be used for preventing, relieving and/or treating cardiotoxicity and cardiac injury caused by chemotherapy drugs without affecting the killing power of chemotherapy drugs against tumors as well as the function of other tissues, thus offering high safety.

### BRIEF DESCRIPTION OF THE DRAWINGS

FIG. 1 is a schematic diagram of the synthesis route of ATBMzyme nanoenzymes.

FIG. 2 shows the TEM detection result of ATBMzyme nanoenzymes.

FIG. 3 shows the result of S, Au, Ru element localization analysis of ATBMzyme nanoenzymes.

FIG. 4 shows the Fourier transform infrared spectroscopy detection result of TBMzyme nanoenzyme, ATBMzyme nanoenzymes, and BMzyme nanoenzyme.

FIGS. 5A and 5B show the XPS detection result of ATBMzyme nanoenzyme, wherein FIG. 5A is the element detection on Au element, and FIG. 5B is the detection on Ru element.

FIG. 6 shows the XRD detection result of TBMzyme nanoenzyme and ATBMzyme nanoenzymes.

FIG. 7 shows the nuclear magnetic hydrogen spectra of TBMzyme nanoenzyme and ATBMzyme nanoenzymes.

FIG. 8 shows the result diagram of ultraviolet-visible light absorption spectral analysis conducted with TBMzyme nanoenzyme, ATBMzyme nanoenzymes, and BMzyme nanoenzyme.

FIGS. 9A˜9D illustrate the detection results of the activity of catalase (CAT) in Au—Ru nanoenzymes, activity of superoxide dismutase (SOD), ABTS radical scavenging ratio, and DPPH radical scavenging ratio in the present disclosure, wherein FIG. 9A shows the detection results of activity of catalase (CAT); FIG. 9B shows the detection results of activity of superoxide dismutase (SOD); FIG. 9C shows the detection results of ABTS radical scavenging ratio; FIG. 9D shows the detection results of DPPH radical scavenging ratio.

FIG. 10 is a radar chart of multiple enzyme activities of Au—Ru nanoenzymes.

FIG. 11 is a schematic diagram of surface configurations of different initial, transition, and final states simulating the catalytic process of Au—Ru nanoenzymes as catalase (CAT).

FIGS. 12A˜12E show the results of detection on enzyme activity and radical scavenging capability of ATBMzyme nanoenzymes, wherein FIG. 12A shows the SOD-like enzyme activity of ATBMzyme nanoenzymes, FIG. 12B shows the specific activity of SOD-like enzyme activity of ATBMzyme nanoenzymes, FIG. 12C shows the CAT-like enzyme activity of ATBMzyme nanoenzymes, FIG. 12D shows the ABTS radical scavenging capability detection on ATBMzyme nanoenzymes, FIG. 12E shows the DPPH radical scavenging capability detection on ATBMzyme nanoenzymes.

FIG. 13 is a representative image of immunofluorescence staining of heart, liver, spleen, lung, kidney, and tumor tissues from tumor-bearing mice; wherein, TBMzyme represents mice in the cy5.5-TBMzyme group, ATBMzyme represents mice in the cy5.5-ATBMzyme group, with a scale bar of 50 μm.

FIG. 14 shows representative HE staining images of liver and kidney from tumor-bearing mice, wherein Vehicle indicates mice in the PBS group, TBMzyme indicates mice in the cy5.5-TBMzyme group, ATBMzyme indicates mice in the cy5.5-ATBMzyme group, with a scale bar of 50 μm.

FIG. 15 shows the detection results of alanine aminotransferase (ALT), aspartate aminotransferase (AST), urea (UREA) and creatinine (CREA) levels in the serum of tumor-bearing mice, wherein Vehicle indicates mice in the PBS group, TBMzyme indicates mice in the cy5.5-TBMzyme group, ATBMzyme indicates mice in the cy5.5-ATBMzyme group; *** indicates p≤0.001 compared with the vehicle group; ## indicates p≤0.01 compared with the TBMzyme group.

FIG. 16 shows a schematic diagram of the medication treatment process for mice in the DOX group, DOX+TBMzyme group, and DOX+ATBMzyme group.

FIGS. 17A˜17C show representative echocardiographic images of cardiac function and quantitative analysis results of ejection fraction and fractional shortening, scale=300 milliseconds (n=6), wherein FIG. 17A shows representative echocardiographic images of cardiac function,

FIG. 17B shows statistical analysis results of EF values, FIG. 17C shows statistical analysis results of FS values; *** indicates p≤0.001 as compared with the vehicle group; # indicates p≤0.05 as compared with the DOX group; ## indicates p≤0.01 as compared with the DOX group; ### indicates p≤0.001 as compared with the DOX group.

FIG. 18 shows the detection results of mice heart injury and heart failure markers LDH, cTNT and CKMB; ** indicates p≤0.01 as compared with the vehicle group; *** indicates p≤0.001 as compared with the vehicle group; # indicates p≤0.05 as compared with the DOX group; ## indicates p≤0.01 as compared with the DOX group; ### indicates p≤0.001 as compared with the DOX group.

FIGS. 19A˜19E show the detection results of the effect of TBMzyme nanoenzyme and ATBMzyme nanoenzymes on tumor killing capability of DOX, wherein FIG. 19A shows the morphology diagram of tumor tissues from four groups of mice; FIG. 19B shows the curve of tumor volume changes over time for four groups of mice; FIG. 19C shows the tumor volumes measured when DOX injection started for four groups of mice; FIG. 19D shows the tumor volumes measured 24 days after DOX injection for four groups of mice; FIG. 19E shows the statistical chart of tumor mass for four groups of mice; *** indicates p<0.001 compared with Vehicle group; # indicates p<0.01 compared with DOX group; ### indicates p<0.001 compared with DOX group.

### DESCRIPTION OF THE EMBODIMENTS

### Example 1

A method of preparing Au—Ru nanoenzymes, with specific steps as follows:
(1) Dissolving HAuCl₄ (0.15 mmol) and RuCl₃·H₂O in 10 milliliters of anhydrous methanol to obtain metal salt solution, wherein the molar ratio of HAuCl₄ to RuCl₃·H₂O is 3:2.
(2) Adding N-acetylcysteine (NAC) and tannic acid (TA) to the metal salt solution prepared in step (1), mixing the mixture uniformly to obtain a mixture, wherein the molar ratio of N-acetylcysteine to HAuCl₄ is 2.7:1; the molar ratio of tannic acid to HAuCl₄ is 2:5.
(3) Adding 4° C. pre-cooled NaBH₄ aqueous solution to the mixture obtained in step (2), stirring and reacting the mixture at room temperature for 1 h, then continue stirring the mixture for 2 h at 60° C. temperature condition; after the reaction is finished, collecting the precipitate by centrifuging at 10000 rpm for 10 min, the precipitate is lyophilized to obtain Au—Ru nanoenzymes (Au—Ru nanoenzymes also referred to as TBMzyme nanoenzyme). The concentration of NaBH₄ aqueous solution is 0.2 mol/L, the molar ratio of NaBH₄ to HAuCl₄ is 1.25:0.15.

### Example 2

A method of preparing Au—Ru nanoenzymes, with specific steps as follows:
(1) Dissolving HAuCl₄ (0.15 mmol) and RuCl₃·H₂O in 10 milliliters of anhydrous methanol to obtain metal salt solution; wherein, the molar ratio of HAuCl₄ to RuCl₃·H₂O is 1.5:2.
(2) Adding N-acetylcysteine (NAC) and tannic acid (TA) to the metal salt solution prepared in step (1), mixing the mixture uniformly to obtain a mixture, wherein the molar ratio of the N-acetylcysteine to HAuCl₄ is 1:1; the molar ratio of the tannic acid to HAuCl₄ is 1:1.
(3) Adding 4° C. pre-cooled NaBH₄ aqueous solution to the mixture obtained in step (2), stirring and reacting the mixture at room temperature for 1 h, then continuing to stir the mixture for 4 h at 50° C. temperature condition; after the reaction is finished, collecting the precipitate by centrifuging at 10000 rpm for 10 min, the precipitate is lyophilized to obtain Au—Ru nanoenzymes (Au—Ru nanoenzymes also referred to as TBMzyme nanoenzyme). The concentration of NaBH₄ aqueous solution is 0.2 mol/L, and the molar ratio of NaBH₄ to HAuCl₄ is 1:0.15.

### Example 3

A method of preparing Au—Ru nanoenzymes, with specific steps as follows:
(1) Dissolving HAuCl₄ (0.15 mmol) and RuCl₃·H₂O in 10 milliliters of anhydrous methanol to obtain metal salt solution; wherein, the molar ratio of HAuCl₄ to RuCl₃·H₂O is 3:1.
(2) Adding N-acetylcysteine (NAC) and tannic acid (TA) to the metal salt solution prepared in step (1), mixing the mixture uniformly to obtain a mixture, wherein the molar ratio of the N-acetylcysteine to HAuCl₄ is 10:1; the molar ratio of the tannic acid to HAuCl₄ is 10:1.
(3) Adding 4° C. pre-cooled NaBH₄ aqueous solution to the mixture obtained in step (2), stirring and reacting the mixture at room temperature for 1 h, then continuing to stir the mixture for 4 h at 60° C. temperature condition; after the reaction is finished, collecting the precipitate by centrifuging at 10000 rpm for 10 min, the precipitate is lyophilized to obtain Au—Ru nanoenzymes (Au—Ru nanoenzymes also referred to as TBMzyme nanoenzyme). The concentration of NaBH₄ aqueous solution is 0.2 mol/L, and the molar ratio of NaBH₄ to HAuCl₄ is 8:0.6.

### Example 4

A method of preparing Au—Ru nanoenzymes, with specific steps as follows:
(1) Dissolving HAuCl₄ (0.15 mmol) and RuCl₃·H₂O in 10 milliliters of anhydrous methanol to obtain metal salt solution; wherein, the molar ratio of HAuCl₄ to RuCl₃·H₂O is 2:1.
(2) Adding N-acetylcysteine (NAC) and tannic acid (TA) to the metal salt solution prepared in step (1), mixing the mixture uniformly to obtain a mixture, wherein the molar ratio of the N-acetylcysteine to HAuCl₄ is 5:1; the molar ratio of the tannic acid to HAuCl₄ is 1:10.
(3) Adding 4° C. pre-cooled NaBH₄ aqueous solution to the mixture obtained in step (2), stirring and reacting the mixture at room temperature for 1 h, then continuing to stir the mixture for 3 h at 60° C. temperature condition; after the reaction is finished, collecting the precipitate by centrifuging at 10000 rpm for 10 min, the precipitate is lyophilized to obtain Au—Ru nanoenzymes (Au—Ru nanoenzymes also referred to as TBMzyme nanoenzyme). The concentration of NaBH₄ aqueous solution is 0.2 mol/L, and the molar ratio of NaBH₄ to HAuCl₄ is 3:0.4.

### Example 5

A method of preparing Au—Ru nanoenzymes, with specific steps as follows:
(1) Dissolving HAuCl₄ (0.15 mmol) and RuCl₃·H₂O in 10 milliliters of anhydrous methanol to obtain metal salt solution; wherein, the molar ratio of HAuCl₄ to RuCl₃·H₂O is 1:1.
(2) Adding N-acetylcysteine (NAC) and tannic acid (TA) to the metal salt solution prepared in step (1), mixing the mixture uniformly to obtain a mixture, wherein the molar ratio of the N-acetylcysteine to HAuCl₄ is 8:1; the molar ratio of the tannic acid to HAuCl₄ is 5:8.
(3) Adding 4° C. pre-cooled NaBH₄ aqueous solution to the mixture obtained in step (2), stirring and reacting the mixture at room temperature for 1 h, then continuing to stir the mixture for 2 h at 60° C. temperature condition; after the reaction is finished, collecting the precipitate by centrifuging at 10000 rpm for 10 min, the precipitate is lyophilized to obtain Au—Ru nanoenzymes (Au—Ru nanoenzymes also referred to as TBMzyme nanoenzyme). The concentration of NaBH₄ aqueous solution is 0.2 mol/L, and the molar ratio of NaBH₄ to HAuCl₄ is 5:0.4.

### Example 6

A method of preparing Au—Ru nanoenzymes, with specific steps as follows:
(1) Dissolving HAuCl₄ (0.15 mmol) and RuCl₃·H₂O in 10 milliliters of anhydrous methanol to obtain metal salt solution; wherein, the molar ratio of HAuCl₄ to RuCl₃·H₂O is 5:2.
(2) Adding N-acetylcysteine (NAC) and tannic acid (TA) to the metal salt solution prepared in step (1), mixing the mixture uniformly to obtain a mixture, wherein the molar ratio of the N-acetylcysteine to HAuCl₄ is 2.7:1; the molar ratio of the tannic acid to HAuCl₄ is 3:1.
(3) Adding 4° C. pre-cooled NaBH₄ aqueous solution to the mixture obtained in step (2), stirring and reacting the mixture at room temperature for 1 h, then continuing to stir the mixture for 2 h at 60° C. temperature condition; after the reaction is finished, collecting the precipitate by centrifuging at 10000 rpm for 10 min, the precipitate is lyophilized to obtain Au—Ru nanoenzymes (Au—Ru nanoenzymes also referred to as TBMzyme nanoenzyme). The concentration of NaBH₄ aqueous solution is 0.2 mol/L, and the molar ratio of NaBH₄ to HAuCl₄ is 1:0.6.

### Example 7

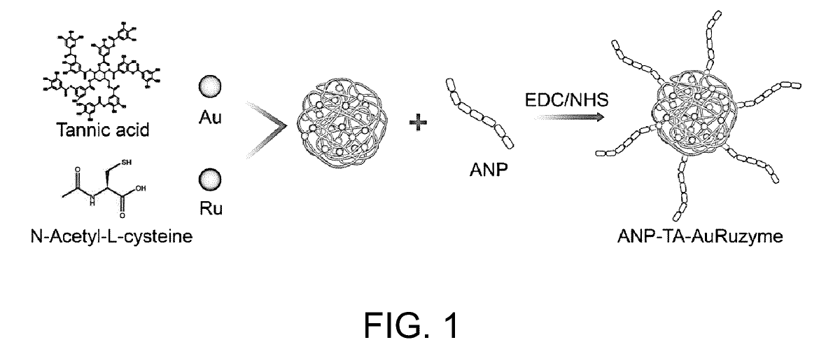

A method of preparing ATBMzyme nanoenzymes (as shown in FIG. 1) specifically includes: dissolving 10 m of Au—Ru nanoenzymes in 2 mL of water to obtain Au—Ru nanoenzymes solution; adding 1-ethyl-3-(3-dimethylaminopropyl) carbodiimide and N-hydroxysuccinimide to the Au—Ru nanoenzymes solution, stirring and reacting the mixture at room temperature for 20 min, then adding brain natriuretic peptide (ANP) to the reaction system, continuing to stir the mixture for 8 h; after the reaction is finished, collecting the precipitate by centrifuging at 10000 rpm for 10 min, the precipitate is lyophilized to obtain ATBMzyme nanoenzymes. The mass ratio of Au—Ru nanoenzymes to 1-ethyl-3-(3-dimethylaminopropyl) carbodiimide is 2:1; the mass ratio of Au—Ru nanoenzymes to N-hydroxysuccinimide is 5:2; the mass ratio of brain natriuretic peptide to Au—Ru nanoenzymes is 1:50; the Au—Ru nanoenzymes is the Au—Ru nanoenzymes prepared in Example 1.

### Example 8

The content of Example 8 is basically the same as Example 7, with the difference being that:

The mass ratio of Au—Ru nanoenzymes to 1-ethyl-3-(3-dimethylaminopropyl)carbodiimide is 1:20; the mass ratio of Au—Ru nanoenzymes to N-hydroxysuccinimide is 1:3; the mass ratio of brain natriuretic peptide to Au—Ru nanoenzymes is 0.2:5; the Au—Ru nanoenzymes is the Au—Ru nanoenzymes prepared in Example 1.

### Example 9

The content of Example 9 is basically the same as Example 7, with the difference being that:

The mass ratio of Au—Ru nanoenzymes to 1-ethyl-3-(3-dimethylaminopropyl)carbodiimide is 4:1; the mass ratio of Au—Ru nanoenzymes to N-hydroxysuccinimide is 5:1; the mass ratio of brain natriuretic peptide to Au—Ru nanoenzymes is 1:5; the Au—Ru nanoenzymes is the Au—Ru nanoenzymes prepared in Example 1.

### Example 10

The content of Example 10 is basically the same as Example 7, with the difference being that:

The mass ratio of Au—Ru nanoenzymes to 1-ethyl-3-(3-dimethylaminopropyl)carbodiimide is 1:10; the mass ratio of Au—Ru nanoenzymes to N-hydroxysuccinimide is 1:1; the mass ratio of brain natriuretic peptide to Au—Ru nanoenzymes is 1:30; the Au—Ru nanoenzymes is the Au—Ru nanoenzymes prepared in Example 2.

### Example 11:

The content of Example 11 is basically the same as Example 7, with the difference being that:

The mass ratio of Au—Ru nanoenzymes to 1-ethyl-3-(3-dimethylaminopropyl)carbodiimide is 3:10; the mass ratio of Au—Ru nanoenzymes to N-hydroxysuccinimide is 5:4; the mass ratio of brain natriuretic peptide to Au—Ru nanoenzymes is 1:50; the Au—Ru nanoenzymes is the Au—Ru nanoenzymes prepared in Example 3.

### Example 12

The content of Example 12 is basically the same as Example 7, with the difference being that:

The mass ratio of Au—Ru nanoenzymes to 1-ethyl-3-(3-dimethylaminopropyl)carbodiimide is 2:1; the mass ratio of Au—Ru nanoenzymes to N-hydroxysuccinimide is 5:2; the mass ratio of brain natriuretic peptide to Au—Ru nanoenzymes is 1:20; the Au—Ru nanoenzymes is the Au—Ru nanoenzymes prepared in Example 4.

### Example 13

The content of Example 13 is basically the same as Example 7, with the difference being that:

The mass ratio of Au—Ru nanoenzymes to 1-ethyl-3-(3-dimethylaminopropyl)carbodiimide is 1:4; the mass ratio of Au—Ru nanoenzymes to N-hydroxysuccinimide is 6:5; the mass ratio of brain natriuretic peptide to Au—Ru nanoenzymes is 1:25; the Au—Ru nanoenzymes is the Au—Ru nanoenzymes prepared in Example 5.

### Example 14

The content of Example 14 is basically the same as Example 7, with the difference being that:

The mass ratio of Au—Ru nanoenzymes to 1-ethyl-3-(3-dimethylaminopropyl)carbodiimide is 1:1; the mass ratio of Au—Ru nanoenzymes to N-hydroxysuccinimide is 4:3; the mass ratio of brain natriuretic peptide to Au—Ru nanoenzymes is 1:10; the Au—Ru nanoenzymes is the Au—Ru nanoenzymes prepared in Example 6.

### (I) Characterization of Au—Ru Nanoenzymes (Also Recorded as TBMzyme Nanoenzymes) and ATBMzyme Nanoenzymes

The Au—Ru nanoenzymes (also referred to as TBMzyme nanoenzyme) prepared in Example 1 and the ATBMzyme nanoenzymes prepared in Example 7 were characterized. At the same time, for comparison, the present disclosure further prepared BMzyme nanoenzyme. The method of preparing BMzyme nanoenzyme is basically the same as Example 1, with the difference being: in step (2), N-acetylcysteine (NAC) was added to the metal salt solution prepared in step (1), and they were mixed uniformly to obtain a mixture.

### 1. Morphology Characterization of Nanoenzymes:

The microscopic morphology of ATBMzyme nanoenzymes was characterized through transmission electron microscopy (TEM), and the result is shown in FIG. 1.

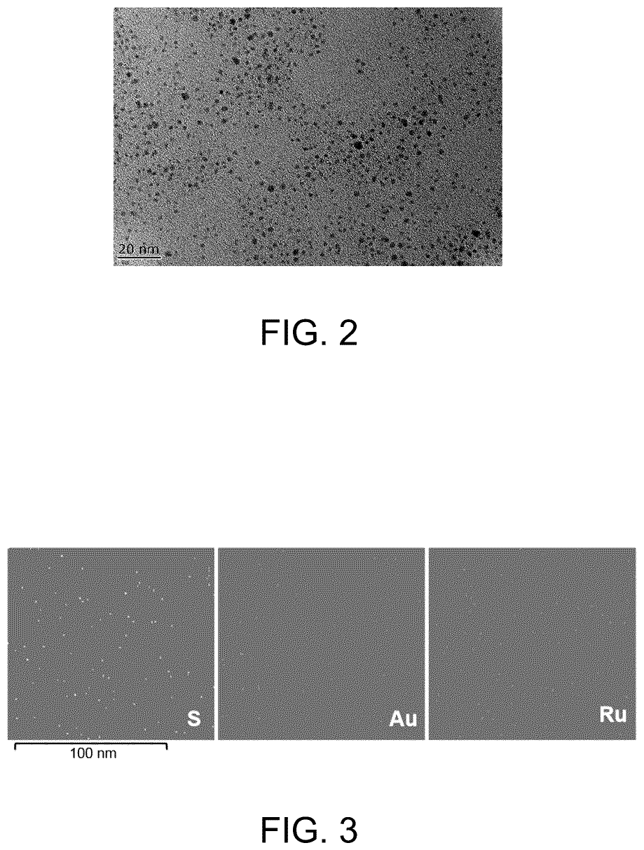

As shown in FIG. 2, ATBMzyme presents uniform spherical nanoparticle morphology, with an average particle size of 3 nm to 5 nm.

### 2. Element Localization Analysis of TBMzyme Nanoenzyme and ATBMzyme Nanoenzymes:

Element Mapping is adopted to conduct element localization analysis for ATBMzyme nanoenzymes, and the result is shown in FIG. 3.

As shown in FIG. 3, the ATBMzyme nanoenzymes contains S, Au, and Ru elements, which proves the successful synthesis of the ATBMzyme nanoenzymes.

### 3. TBMzyme Nanoenzyme, ATBMzyme Nanoenzymes Fourier Transform Infrared Spectroscopy (FTIR) Analysis:

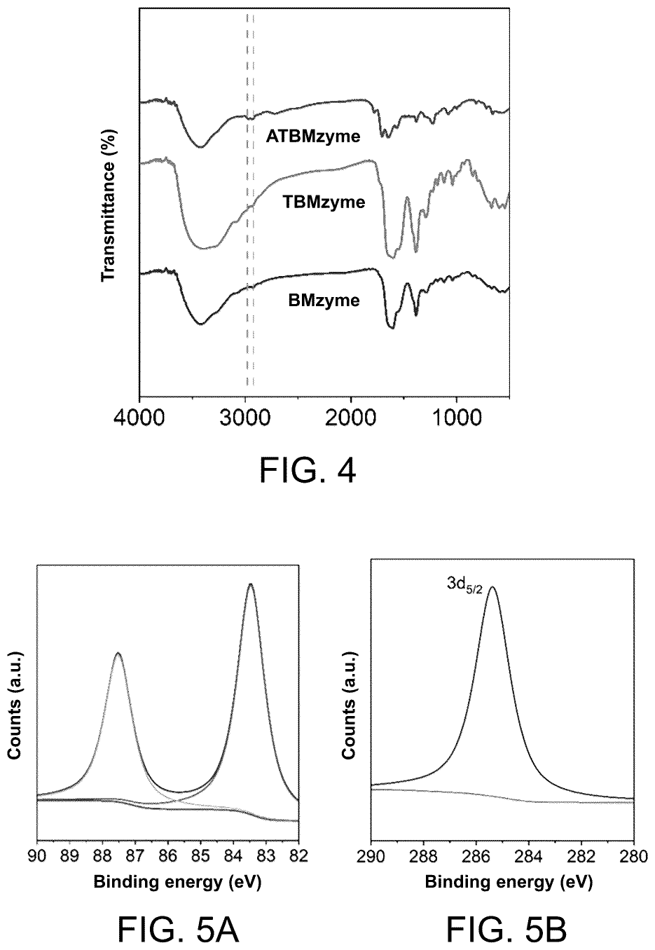

Fourier transform infrared spectroscopy (FTIR) detection was conducted on TBMzyme nanoenzyme, ATBMzyme nanoenzymes, and BMzyme nanoenzyme, with the results shown in FIG. 4.

FIG. 4 shows that ANP was successfully conjugated to the TBMzyme nanoenzyme, providing proof of the synthesis of ATBMzyme.

### 4. ATBMzyme Nanoenzymes X-Ray Photoelectron Spectroscopy (XPS) Analysis:

X-ray photoelectron spectroscopy (XPS) detection was conducted on ATBMzyme nanoenzymes, and the result is shown in FIGS. 5A and 5B.

FIGS. 5A and 5B show that the high resolution XPS spectrum of Au orbital shows peaks corresponding to zero valence Au, with binding energy at 83.7 eV. In addition, at the binding energy of 285.4 eV, the XPS spectrum of Ru orbital shows peaks corresponding to Ru 3d5/2, which further proves the presence of oxidized Ru on ATBMzyme.

### 5. X-Ray Diffraction (XRD) Analysis of the Nanoenzymes:

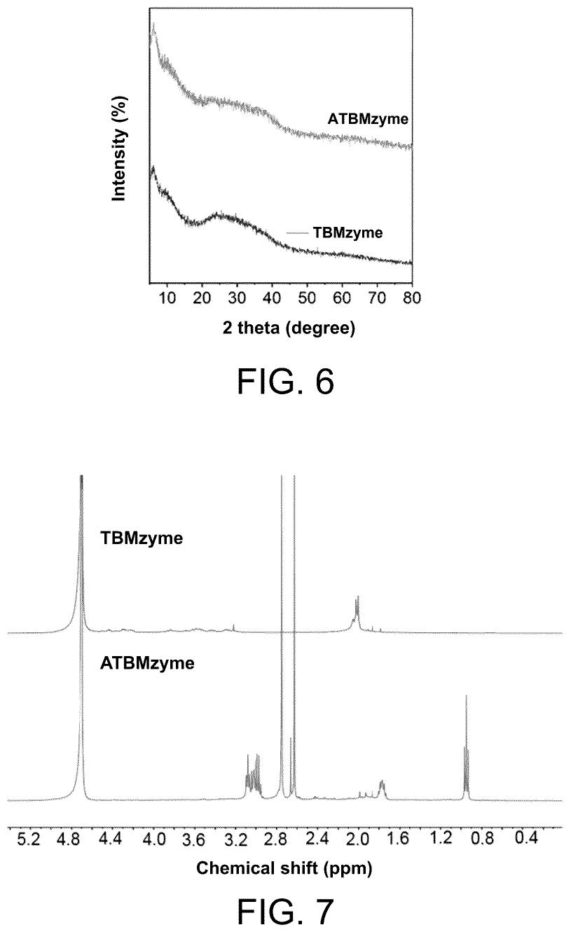

XRD detection was conducted on TBMzyme nanoenzyme and ATBMzyme nanoenzymes, the detection result is shown in FIG. 6.

FIG. 6 shows that TBMzyme nanoenzyme and ATBMzyme exist in amorphous form.

### 7. Nuclear Magnetic Analysis on TBMzyme Nanoenzyme and ATBMzyme Nanoenzymes:

Nuclear magnetic detection was conducted on TBMzyme nanoenzyme and ATBMzyme nanoenzymes, and the results are shown in FIG. 7.

As shown in FIG. 7, the characteristic peak of ANP appears in the ATBMzym spectrum, providing proof of successful conjugation of ANP.

### 8. Ultraviolet-Visible Absorption Spectral Analysis on TBMzyme Nanoenzyme and ATBMzyme Nanoenzymes:

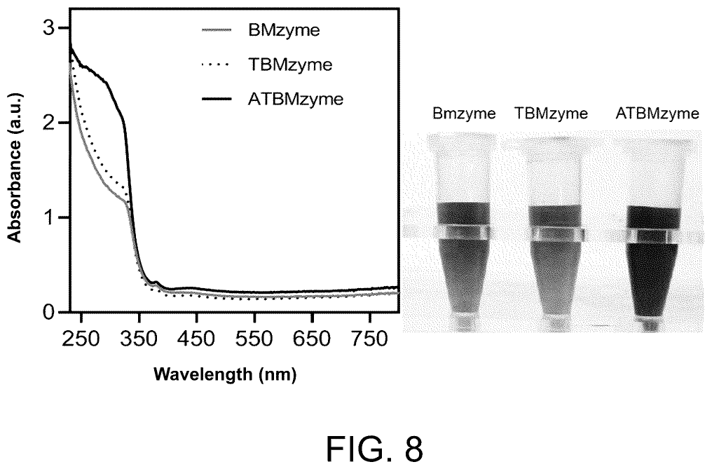

Ultraviolet-visible light absorption spectral analysis was conducted on TBMzyme nanoenzyme and ATBMzyme nanoenzymes, and the results are shown in FIG. 8.

As known from FIG. 8, BMzyme, TBMzyme nanoenzymes and ATBMzyme present good stability and solubility uniformity.

### (II) Enzyme Activity and Radical Scavenging Capability Detection on Au—Ru Nanoenzyme (TBMzyme Nanoenzyme)

Taking the Au—Ru nanoenzymes prepared in Example 1 as an example, enzyme activity and radical scavenging capability detection are conducted. At the same time, for comparison with the Au—Ru nanoenzymes prepared by the present disclosure, the present disclosure further prepares Au—Ru nanoenzymes, Au—Fe nanoenzyme, Au—Mn nanoenzyme, Au—Cu nanoenzyme, Ru—Fe nanoenzyme, Mn—Fe nanoenzyme, Fe—Cu nanoenzyme, Cu—Mn nanoenzyme, Ru—Mn nanoenzyme, and Ru—Cu nanoenzyme. The method of preparing Au—Ru nanoenzymes, Au—Fe nanoenzyme, Au—Mn nanoenzyme, Au—Cu nanoenzyme, Ru—Fe nanoenzyme, Mn—Fe nanoenzyme, Fe—Cu nanoenzyme, Cu—Mn nanoenzyme, Ru—Mn nanoenzyme, Ru—Cu nanoenzyme is basically the same as Example 1, with the difference being that: replacing HAuCl₄ and ruthenium metal salt in step (1) with metal salts corresponding to the metal elements in the nanoenzyme.

### 1. CAT Enzyme Activity Detection on Au—Ru Nanoenzymes:

CAT enzyme, also known as hydrogen peroxide enzyme, may decompose H₂O₂ to produce O₂ and water. The method for detecting CAT enzyme activity is: preparing the nanoenzyme into 1 mg/mL aqueous solution, and dispersing it with a volume of 200 ul into 10 mL of aqueous solution containing 0.1 mM hydrogen peroxide (H₂O₂), and using dissolved oxygen meter to measure the production of O₂.

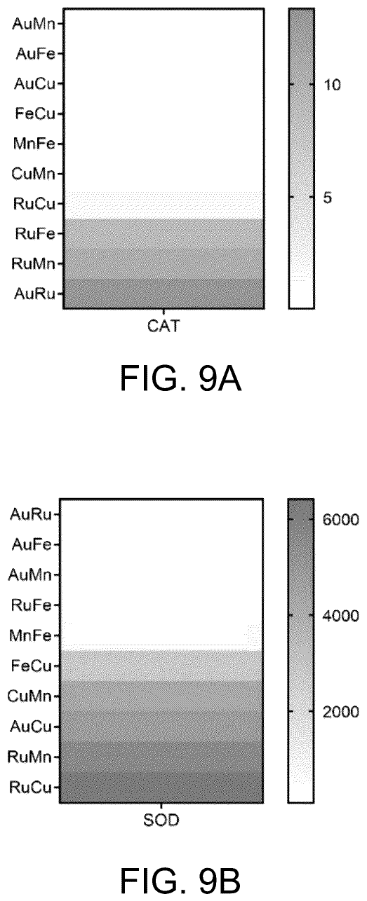

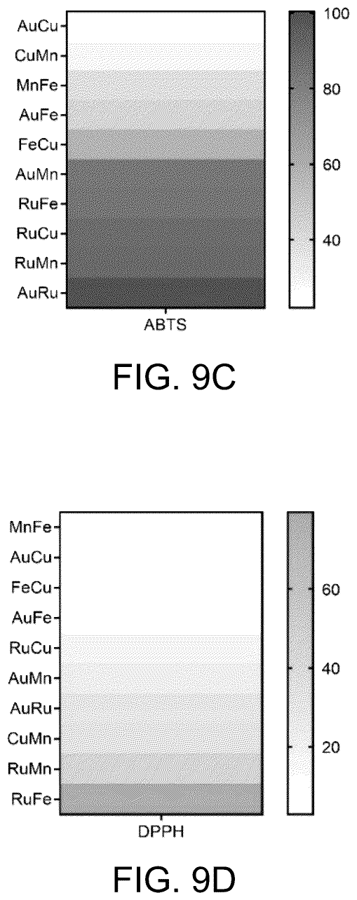

CAT enzyme activity detection result is shown in FIG. 9A.

As may be known in FIG. 9A, the CAT enzyme of Au—Ru nanoenzymes exhibits the highest activity.

### 2. SOD Enzyme Activity Detection on Au—Ru Nanoenzymes:

SOD enzyme, also known as superoxide dismutase, may dismutate superoxide anions to produce H₂O₂ and H₂O. The SOD mimetic enzyme activity of the sample was determined using the xanthine oxidase method. The specific operation is as follows: the prepared nanoenzyme solution (0.0015625˜0.1 mg/mL) was added to a 96-well plate with a volume of 30 μL, followed by the addition of xanthine, pH 7.4 phosphate buffer, cytochrome C, xanthine oxidase working solution and water. An enzyme microplate reader was used to measure the absorption of light at 450 nm for each well 1 minute before and after the addition, and the inhibition percentage was calculated. The detection result is shown in FIG. 9B.

As may be known in FIG. 9B, the activity of SOD mimetic enzyme of Ru—Cu nanoenzyme is the highest, and the activity of SOD enzyme of Au—Ru nanoenzymes is relatively low.

### 3. Detection on ABTS Radical Scavenging Activity of Au—Ru Nanoenzymes:

ABTS after oxidation may produce structurally stable blue-green ABTS radical, which has a maximum absorption peak at 735 nm. After ABTS reacts with oxidants, it will produce blue-green ABTS cation radical (ABTS⋅+), generating a characteristic absorption peak at 734 nm wavelength. When antioxidants are present, the production of ABTS⋅+ will be inhibited, causing the absorption peak at 735 nm to decrease, and the degree of decrease in the absorption peak is proportional to the degree of radical scavenging. Therefore, the present disclosure adopts the ABTS method to detect the radical scavenging activity of Au—Ru nanoenzymes. The specific experimental operation is as follows. First, ABTS solution (7 mM) and potassium persulfate (2.45 mM) are incubated overnight to activate ABTS radical. The bimetallic nanoenzymes (Au—Ru nanoenzymes, Au—Fe nanoenzyme, Au—Mn nanoenzyme, Au—Cu nanoenzyme, Ru—Fe nanoenzyme, Mn—Fe nanoenzyme, Fe—Cu nanoenzyme, Cu—Mn nanoenzyme, Ru—Mn nanoenzyme, Ru—Cu nanoenzyme) with final concentrations of (1.5625˜100 μg/mL) are added to the ABTS radical solution. Within 6 minutes, the time course absorption of light of ABTS radical at 734 nm is measured. ABTS scavenging percentage=(A0−Asample)/A0*100%. A0 is the absorption of light of ABTS at 520 nm without addition of sample; Asample is the absorption of light of ABTS at 520 nm after addition of the sample. The detection results are shown in FIG. 9C.

As known from FIG. 9C, Au—Ru nanoenzymes show the highest capability to scavenge ABTS radicals.

### 4. Detection on DPPH Radical Scavenging Activity of Au—Ru Nanoenzymes:

DPPH is a stable radical, easily soluble in polar solvents such as methanol, ethanol, and so on, and has a maximum absorption peak at 520 nm. When antioxidants are added, DPPH undergoes a decolorization reaction. The specific operation for DPPH radical scavenging activity determination is as follows: dissolving DPPH in anhydrous ethanol, keeping it away from light, then mixing the prepared DPPH solution with different concentrations of bimetallic nanoenzymes (Au—Ru nanoenzymes, Au—Fe nanoenzyme, Au—Mn nanoenzyme, Au—Cu nanoenzyme, Ru—Fe nanoenzyme, Mn—Fe nanoenzyme, Fe—Cu nanoenzyme, Cu—Mn nanoenzyme, Ru—Mn nanoenzyme, Ru—Cu nanoenzyme) at (1.5625˜100 μg/mL), incubating it in darkness at 37° C. for 30 minutes, then recording the absorption of light at 520 nm using an enzyme-labeled instrument, and calculating the scavenged DPPH. The detection results are shown in FIG. 9D.

As known from FIG. 9D, Ru—Fe exhibits the highest capability to scavenge DPPH radical, while Au—Ru also possesses relatively high DPPH radical scavenging capability.

### 5. Radar Chart Analysis on Multiple Enzyme Activities of Metal Nanoenzymes

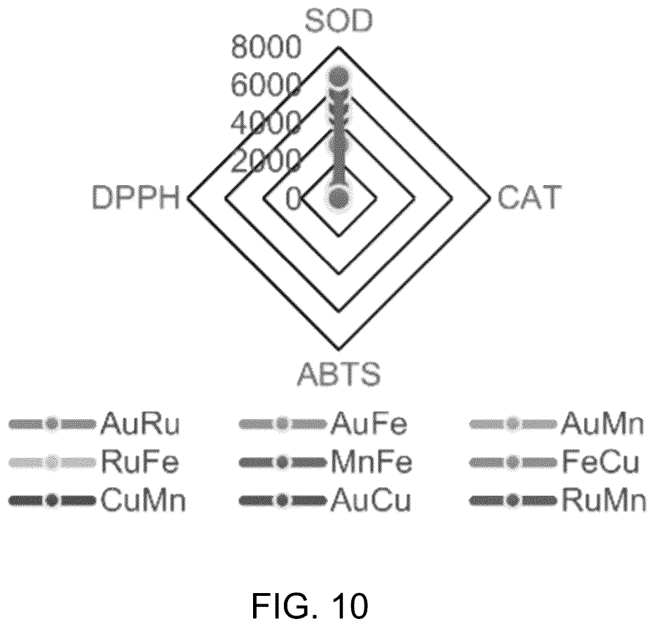

Multi-class enzyme activity radar chart analysis was conducted on the enzyme activity and radical scavenging capability detection data of Au—Ru nanoenzymes, Au—Fe nanoenzyme, Au—Mn nanoenzyme, Au—Cu nanoenzyme, Ru—Fe nanoenzyme, Mn—Fe nanoenzyme, Fe—Cu nanoenzyme, Cu—Mn nanoenzyme, Ru—Mn nanoenzyme, and Ru—Cu nanoenzyme, and the results are shown in FIG. 10.

As may be known from FIG. 10, Au—Ru bimetallic nanoenzymes possess the optimal synergistic antioxidant activity

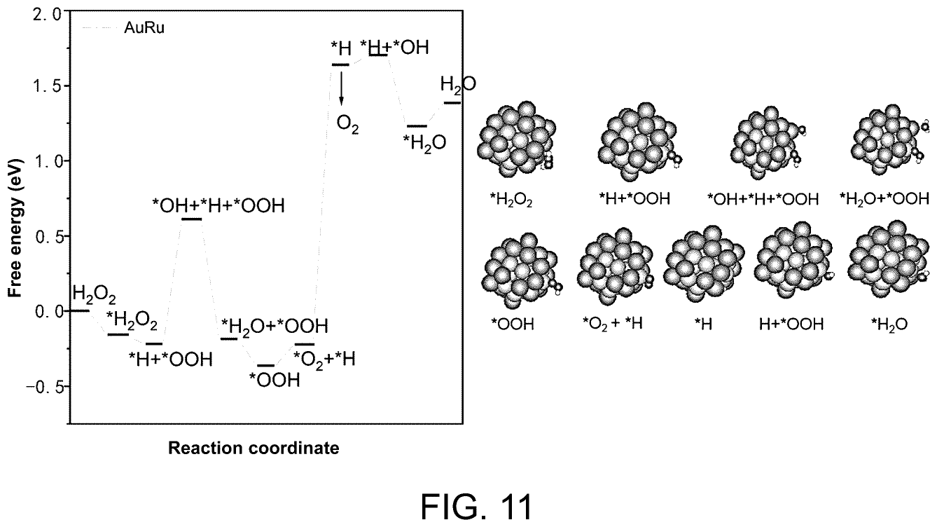

The catalytic process surface configuration of Au—Ru nanoenzymes of the present disclosure catalyzing the decomposition of H₂O₂ is shown in FIG. 11.

From FIG. 11, it may be known that Au—Ru nanoenzymes catalytic decomposition of H₂O mainly includes two processes. The first process is the initial adsorption of OH—; the second process is the desorption of O₂. The energy barrier for O₂ desorption is very high, indicating that O₂ produced in the decomposition process on the pure alloy surface easily oxidizes the alloy. This indicates that the alloy has high reducing capability, making the production of initial O₂ challenging. In the actual reaction, small alloy particles are oxidized by H₂O₂. Only when they are oxidized to a specific degree, their reducing capability weakens, thus making the production of O₂ easier. The energy barrier for initial OH— adsorption is not particularly high (even lower for the second OH— adsorption). Electron and proton conjugating reactions may also directly produce H₂O through the Eley-Rideal mechanism.

The above research shows that Au—Ru nanoenzymes may have the potential to scavenge reactive oxygen species (ROS) and reactive nitrogen species (RNS). Therefore, Au—Ru nanoenzymes were selected as the experimental drug, and their antioxidant effects were further verified in vivo.

### (III) Detection on Enzyme Activity and Radical Scavenging Capability of ATBMzyme Nanoenzyme

Taking the ATBMzyme nanoenzymes prepared in Example 7 as an example, enzyme activity and radical scavenging capability detection were conducted.

### 1. Detection on SOD Enzyme Activity of ATBMzyme Nanoenzymes:

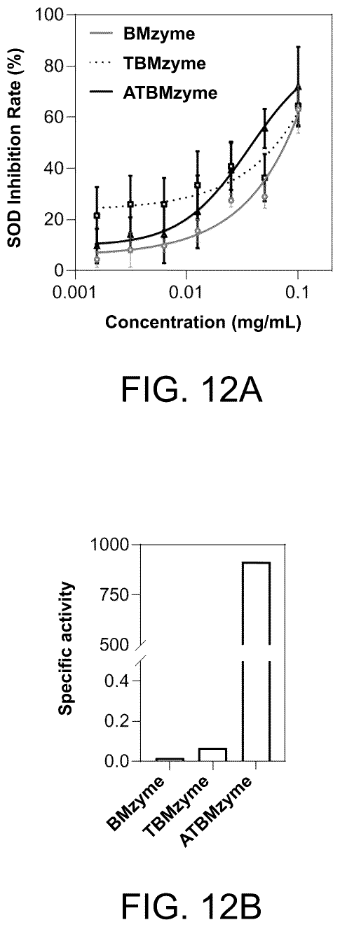

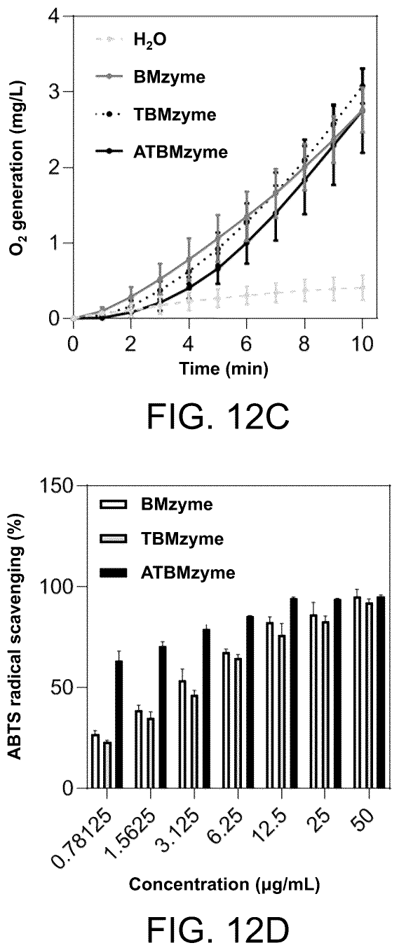

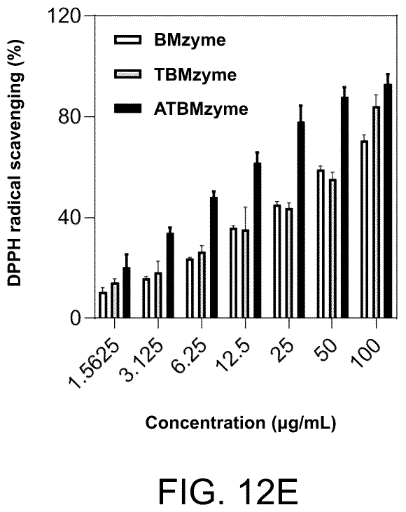

SOD enzyme, also known as superoxide dismutase, may dismutate superoxide anions to produce H₂O₂ and H₂O. The SOD mimetic enzyme activity of the sample was determined using the xanthine oxidase method. The specific operation is as follows: the prepared nanoenzyme solution (0.0015625˜0.1 mg/mL) was added to a 96-well plate with a volume of 30 μL, followed by the addition of xanthine, pH 7.4 phosphate buffer, cytochrome C, xanthine oxidase working solution and water. An enzyme marker was used to measure the absorption of light at 450 nm for each well 1 minute before and after addition, and the inhibition percentage was calculated. The detection results are shown in FIGS. 12A and 12B.

From FIGS. 12A and 12B, it may be known that TBMzyme and ATBMzyme exhibit superoxide dismutase SOD mimetic enzyme activity in phosphate buffer at pH 7.4, and ATBMzyme has the highest SOD enzyme activity, which is quantitatively determined through theoretical calculation to be approximately 913 U/mg, while BMzyme (0.01644 U/mg) and TBMzyme (0.065 U/mg) have relatively lower SOD enzyme activity. This indicates that the inclusion of ANP ingredient may provide more active sites, enhancing antioxidant capability, while not affecting the capability of nanoenzyme to remove hydrogen peroxide. Therefore, ATBMzyme may remove O₂·⁻, degrade H₂O₂, and promote the production of O₂.

2. Detection on CAT enzyme activity of ATBMzyme nanoenzymes:

CAT enzyme, also known as hydrogen peroxide enzyme, can decompose H₂O₂ to produce O₂ and water. The method for detecting CAT enzyme activity is: preparing the nanoenzyme into 1 mg/mL aqueous solution, and dispersing it with a volume of 200 ul into 10 mL aqueous solution containing 0.1 mM hydrogen peroxide (H₂O₂), and using dissolved oxygen meter to measure the production of O₂.

CAT enzyme activity detection result is shown in FIG. 12C.

As shown in FIG. 12C, ATBMzyme nanoenzyme, TBMzyme nanoenzyme, and BMzyme nanoenzyme exhibit hydrogen peroxide CAT mimetic enzyme activity in phosphate buffer at pH 7.4.

### 3. Detection on ABTS Radical Scavenging Activity of ATBMzyme Nanoenzymes:

ABTS may produce structurally stable blue-green ABTS radical through oxidation, which has a maximum absorption peak at 735 nm. After ABTS reacts with oxidants, it will produce blue-green ABTS cation radical (ABTS⋅+), generating a characteristic absorption peak at 734 nm wavelength. When antioxidants are present, the production of ABTS⋅+ will be inhibited, causing the absorption peak at 735 nm to decrease, and the degree of decrease in the absorption peak is proportional to the degree of radical scavenging. Therefore, the present disclosure adopts the ABTS method to detect the radical scavenging activity of nanoenzymes. The specific experimental operation is as follows: first, ABTS solution (7 mM) is incubated with potassium persulfate (2.45 mM) overnight to activate ABTS radical. Nanoenzymes with final concentrations of (1.5625˜100 μg/mL) are added to the ABTS radical solution. Within 6 minutes, the time course absorption of light of ABTS radical at 734 nm is measured. ABTS scavenging percentage=(A0−Asample)/A0*100%. A0 is the absorption of light of ABTS at 520 nm without adding sample; Asample is the absorption of light of ABTS at 520 nm after adding sample. The detection results are shown in FIG. 12D.

As known from FIG. 12D, ATBMzyme nanoenzyme, TBMzyme nanoenzyme, BMzyme nanoenzyme significantly inhibited the production of ABTS radical, with ATBMzyme showing the strongest scavenging effect. These results show that ATBMzyme nanoenzyme, TBMzyme nanoenzyme, BMzyme nanoenzyme may be used for the treatment of diseases related to nitrogen radical.

### 4. Detection on DPPH Radical Scavenging Activity of ATBMzyme Nanoenzymes:

DPPH is a stable radical, easily soluble in methanol, ethanol and other polar solvents, and has a maximum absorption peak at 520 nm. When antioxidants are added, DPPH undergoes a decolorization reaction. The specific operation of measuring DPPH radical scavenging activity is: dissolving DPPH in anhydrous ethanol, keeping it away from light, and then mixing the prepared DPPH solution with nanoenzymes of different concentrations, incubating it in the dark at 37° C. for 30 minutes, then recording its absorption of light at 520 nm using an enzyme marker, and calculating the scavenged DPPH. The detection results are shown in E of FIG. 12.

As known from FIG. 12E, ATBMzyme nanoenzymes and TBMzyme nanoenzyme significantly inhibited the production of DPPH hydroxyl radical, with ATBMzyme nanoenzymes showing the strongest scavenging effect.

The above enzyme activity and radical scavenging capability experiment results show that ANP and TA both serve a synergistic function, jointly regulating the enzyme activity of ATBMzyme nanoenzymes, and have an important effect on the enzyme activity of ATBMzyme nanoenzymes.

### (IV) In Vivo Distribution Study and Safety Evaluation of TBMzyme Nanoenzyme and ATBMzyme Nanoenzymes

### 1. Experimental Animals

Eight-week-old male balb/c mice were obtained from the Institute of Laboratory Animal Science, Chinese Academy of Medical Sciences (Beijing, China), and were adaptively fed for one week before the start of the research. All mice were placed in a temperature-controlled environment, with a 12-hour light/dark cycle at 22±2° C., and were provided with sterile food and drinking water. All experiments involving animals were conducted according to the “Guide for the Use and Care of Laboratory Animals”, and this research was approved by the Animal Care and Use Committee Sub-center of the National Center for Cardiovascular Diseases (FZX-IACUCC-2024005).

### 2. Construction of Tumor-Bearing Mice Model:

Eight-week-old balb/c mice, after removing the hair under the armpit, were subcutaneously injected with 8×10⁶ 4T1 cells, and small rounded protruding vesicles could be observed at the inoculation site. The mice were returned to the cage, and after 3-5 days, obvious white masses could be seen at the injection site, indicating the successful establishment of the tumor-bearing mice model.

### 3. Experimental Animal Grouping and Processing

Eighteen tumor-bearing mice were divided into PBS group, cy5.5-TBMzyme group and cy5.5-ATBMzyme group, with six mice in each group.

### 4. Experimental Drugs:

The test drugs are: Cy5.5 labeled ATBMzyme nanoenzymes (cy5.5-ATBMzyme nanoenzymes), Cy5.5 labeled TBMzyme (cy5.5-TBMzyme nanoenzyme).

Synthesis of cy5.5-ATBMzyme nanoenzymes: 10 mg of TBMzyme/ATBMzyme were weighed and dispersed in 10 ml of deionized water, 1 mg of Cy5.5-NHS ester were added and stirred for 2 h, and dialyzed and lyophilized to obtain Cy5.5 labeled ATBMzyme nanoenzymes. cy5.5-TBMzyme nanoenzyme is the same as cy5.5-ATBMzyme nanoenzymes. The method of preparing cy5.5-ATBMzyme nanoenzymes solution is: weighing appropriate amount of cy5.5-ATBMzyme nanoenzymes lyophilized powder, adding appropriate amount of deionized water, and preparing 5 mg/mL of cy5.5-ATBMzyme nanoenzymes solution using probe ultrasonication. The method of preparing cy5.5-TBMzyme nanoenzyme solution is the same as that for cy5.5-ATBMzyme nanoenzymes solution.

### 5. In Vivo Distribution Study of TBMzyme Nanoenzyme and ATBMzyme Nanoenzymes:

Through tail vein injection, PBS buffer, cy5.5-ATBMzyme nanoenzymes solution, and cy5.5-TBMzyme nanoenzyme solution were injected into tumor-bearing mice in PBS group, cy5.5-TBMzyme group, and cy5.5-ATBMzyme group respectively, with an injection dose of 10 mg/kg body weight. At 24 h after injection, the heart, liver, spleen, kidney, lung, and tumor tissues of the mice were collected for immunofluorescence staining of frozen sections.

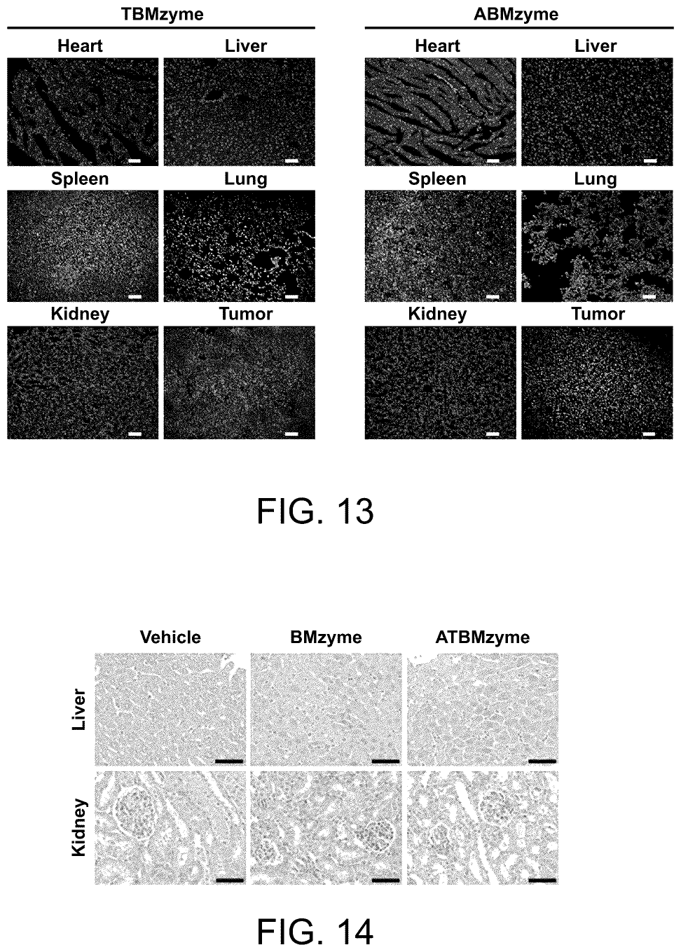

The experimental results are shown in FIG. 13.

As known from FIG. 13, both ATBMzyme nanoenzyme and TBMzyme nanoenzyme distribution can be detected in heart tissue, while there is relatively less distribution in other tissues, and the distribution of ATBMzyme nanoenzyme in heart tissue is more than that of TBMzyme nanoenzyme. The results show that both ATBMzyme nanoenzyme and TBMzyme nanoenzyme may target heart tissue.

### 6. Biological Safety Assessment of TBMzyme Nanoenzyme and ATBMzyme Nanoenzyme:

Through tail vein injection, PBS buffer, cy5.5-ATBMzyme nanoenzymes solution, and cy5.5-TBMzyme nanoenzyme solution were injected into tumor-bearing mice in PBS group, cy5.5-TBMzyme group, and cy5.5-ATBMzyme group, respectively, with an injection dose of 10 mg/kg body weight. The injection was performed once a week for a total of 4 weeks. After 4 weeks, the liver and kidney tissues of the mice were collected for HE staining. Meanwhile, blood samples were collected from the mice by orbital blood collection method, and the blood samples were centrifuged at 3000 rpm for 10 minutes. Then, serum was collected from the supernatant, and ELISA kits were used to evaluate the levels of alanine aminotransferase (ALT, elabscience, E-BC-K235-M), aspartate aminotransferase (AST, elabscience, E-BC-K236-M), urea (UREA, elabscience, E-BC-K183-M), and creatinine (CREA, elabscience, E-BC-K188-M) in the serum.

The HE staining results of liver and kidney tissues of mice are shown in FIG. 14.

As shown in FIG. 14, it may be known that there were no significant differences in the morphology of liver tissue and kidney tissue of tumor-bearing mice among the PBS group, cy5.5-TBMzyme group, and cy5.5-ATBMzyme group.

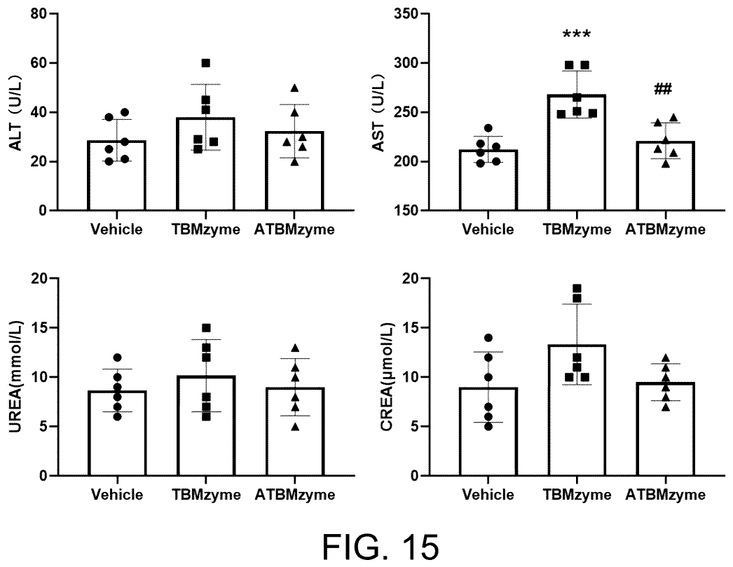

The detection results of alanine aminotransferase (ALT), aspartate aminotransferase (AST), urea (UREA) and creatinine (CREA) levels in the serum of tumor-bearing mice are shown in FIG. 15.

As shown in FIG. 15, there are no significant differences in the levels of alanine aminotransferase (ALT), aspartate aminotransferase (AST), urea (UREA), and creatinine (CREA) in the serum of tumor-bearing mice among the PBS group, cy5.5-TBMzyme group, and cy5.5-ATBMzyme group, indicating that TBMzyme nanoenzyme and ATBMzyme nanoenzymes do not affect the liver and kidney functions of mice.

The above results show that TBMzyme nanoenzyme and ATBMzyme nanoenzymes may target the heart tissue to exert their effects, and at the same time, they do not affect the function of other tissues while exerting their effects, indicating good safety.

### (V) Effects of TBMzyme Nanoenzyme and ATBMzyme Nanoenzymes on DOX-Induced Myocardial Injury and Tumor-Killing Effect of DOX

### 1. Experimental Animals:

Eight-week-old male balb/c mice were obtained from the Institute of Laboratory Animal Science, Chinese Academy of Medical Sciences (Beijing, China), and were adaptively fed for one week before the start of the research. All mice were placed in a temperature-controlled environment, with a 12-hour light/dark cycle at 22±2° C., and were provided with sterile food and drinking water. All experiments involving animals were conducted according to the “Guide for the Use and Care of Laboratory Animals”, and this research was approved by the Animal Care and Use Committee Sub-center of the National Center for Cardiovascular Diseases (FZX-IACUCC-2024005).

### 2. Construction of Tumor-Bearing Mice Model:

The method for constructing tumor-bearing mice models is the same as in Example 6, which will not be repeated here. After the successful construction of the tumor-bearing mice models, a vernier caliper was used to measure the length and width of the tumor mass, and the volume of the tumor mass was calculated.

### 3. Experimental Animal Grouping and Processing

Twenty-four tumor-bearing mice were divided into Vehicle group, DOX group, DOX+TBMzyme group, and DOX+ATBMzyme group, with 6 mice in each group.

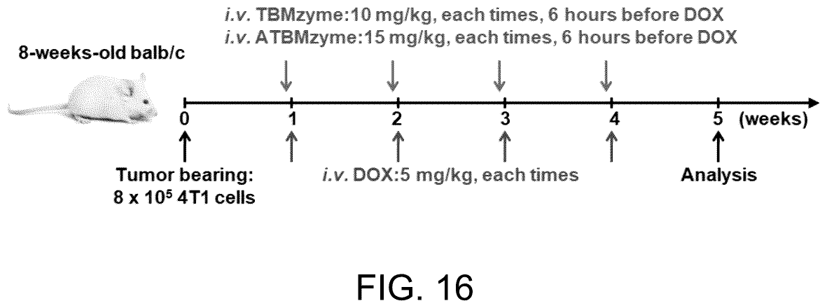

Tumor-bearing mice in DOX group, DOX+TBMzyme group, and DOX+ATBMzyme group were all injected with DOX through tail vein (injection dose was 5 mg/kg body weight). Mice of the Vehicle group were injected with saline through tail vein, with injections once per week for a total of four weeks. Moreover, 6 h before each DOX injection, tumor-bearing mice of the DOX group, DOX+TBMzyme group, and DOX+ATBMzyme group were injected through tail vein with saline (injection dose was 1 ml/kg body weight), TBMzyme nanoenzyme solution (injection dose was 10 mg/kg body weight), ATBMzyme nanoenzymes solution (injection dose was 15 mg/kg body weight) respectively. Mice of Vehicle group were injected with saline through tail vein at the same time period. The TBMzyme nanoenzyme solution and ATBMzyme nanoenzymes solution used for injection were both prepared using water as solvent, with a concentration of 2.5 mg/mL (the schematic diagram of drug treatment process for DOX group, DOX+TBMzyme group, and DOX+ATBMzyme group mice is shown in FIG. 16).

### 4. Ultrasound Echocardiography of Mice:

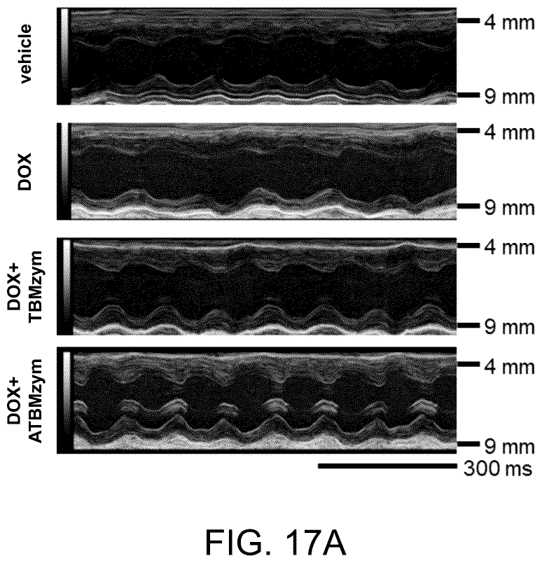

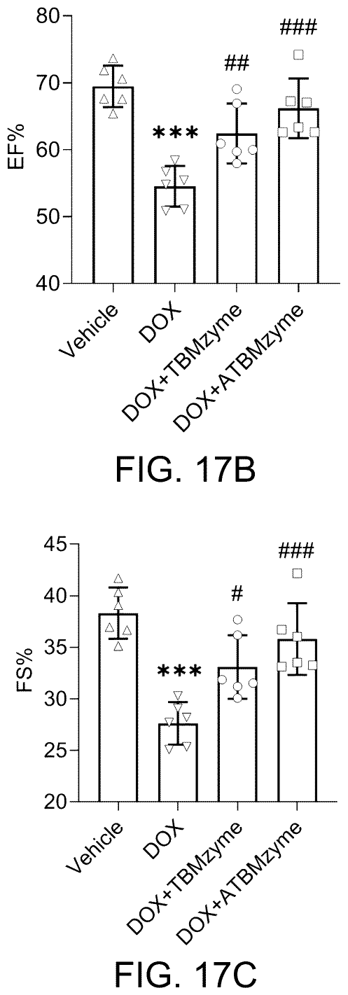

Using Visual Sonics Vevo 3000 system (Fuji Visual Sonics, Japan), transthoracic echocardiography was conducted on mice in the Vehicle group, DOX group, DOX+TBMzyme group, and DOX+ATBMzyme group. During the examination, mice were anesthetized and maintained in an environment with 1-2% isoflurane and 2 liters per minute of 20% oxygen. Vevo analysis software (Fuji Visual Sonics, Japan) was used to analyze the left ventricular ejection fraction (EF) and left ventricular fractional shortening (FS). The detection results are shown in FIG. 17.

As shown in FIG. 17, DOX significantly weakens the cardiac ejection function in mice, while TBMzyme and ATBMzyme may effectively relieve the cardiac function impairment caused by DOX.

### 5. Detection on Myocardial Enzyme Profile in Mice:

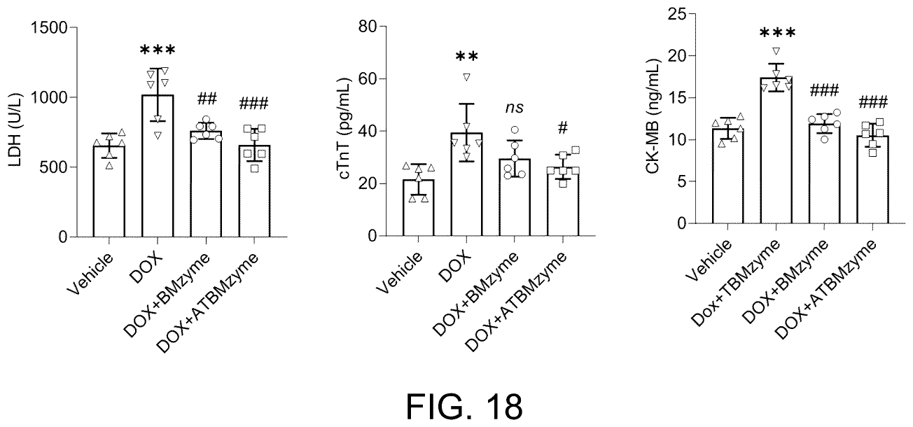

For Vehicle group, DOX group, DOX+TBMzyme group, and DOX+ATBMzyme group mice, detection of cardiac injury and heart failure markers LDH, cTNT, and CKMB was conducted. LDH, cTNT, and CKMB were all detected using ELISA kits, wherein, for LDH, the ELISA kit catalog number was No. E-EL-M0419c, purchased from Elabscience company; for cTNT, the ELISA kit catalog number was No. E-EL-M1801c, purchased from Elabscience company; for CKMB, the ELISA kit catalog number was No. E-EL-M0355c, purchased from Elabscience company. The detection results are shown in FIG. 18.

As shown in FIG. 18, it may be known that the supplementation of TBMzyme and ATBMzyme may effectively relieve the elevation of myocardial enzyme profile caused by DOX, reducing myocardial cell damage.

### 6. The Influence of TBMzyme Nanoenzyme and ATBMzyme Nanoenzymes on the Tumor Killing Effect of DOX:

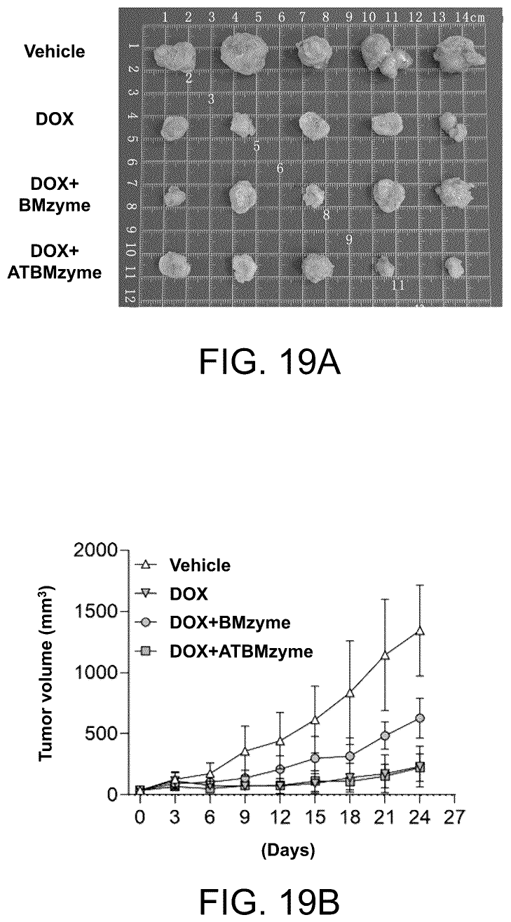

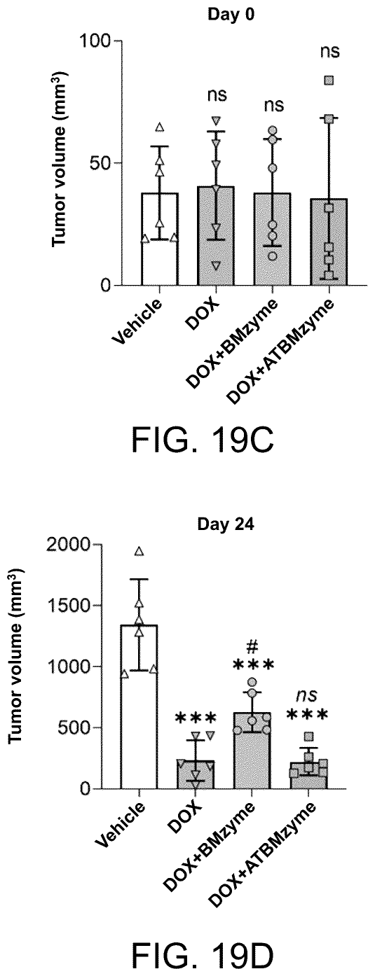

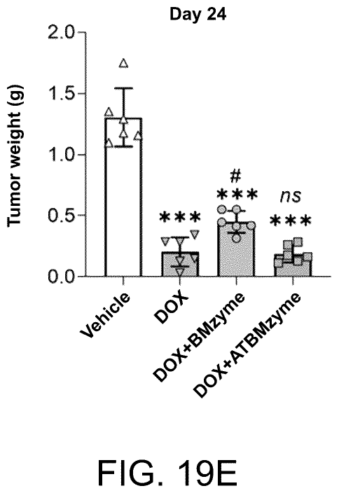

To study whether ATBMzyme affects the tumor-killing effect of DOX, tumor tissues were taken from tumor-bearing mice in the Vehicle group, DOX group, DOX+TBMzyme group, and DOX+ATBMzyme group, respectively to observe changes in tumor tissue size. At the same time, on the first day of DOX injection (designated as Day 0) and the 24th day (designated as Day 24), the tumor volume and weight of the four groups of mice were measured and calculated. The experimental results are shown in FIGS. 19A˜19E.

As shown in FIGS. 19A˜19E, compared with the DOX group, there was no difference in tumor tissue size in the DOX+TBMzyme group and DOX+ATBMzyme group, while these three groups showed significantly smaller tumor volume compared with the Vehicle group, indicating that the nanoenzymes do not affect the tumor size after DOX treatment. The results show that ATBMzyme does not affect the killing effect of DOX on tumors.

What is claimed is:

1. A method for preparing Au—Ru nanoenzymes, comprising the following steps:
(1) dissolving HAuCl₄ and ruthenium metal salt in a solvent to obtain a metal salt solution;
(2) adding N-acetylcysteine and tannic acid to the metal salt solution, mixing uniformly to obtain a mixture;
(3) adding NaBH₄ aqueous solution to the mixture, stirring and reacting the mixture at room temperature for 0.5 h˜2 h, then continuing to stir and react the mixture at 50° C.˜70° C. for 2 h˜4 h; after the reaction is finished, collecting precipitate by centrifuging, then the precipitate is lyophilized to obtain the Au—Ru nanoenzymes.

2. The method for preparing Au—Ru nanoenzymes according to claim 1, wherein a molar ratio of the HAuCl₄ to the ruthenium metal salt is (1.5˜6):2.

3. The method for preparing Au—Ru nanoenzymes according to claim 1, wherein a molar ratio of the N-acetylcysteine to the HAuCl₄ is (1˜10):1, a molar ratio of the tannic acid to the HAuCl₄ is (1˜10):(1˜10), and a molar ratio of the NaBH₄ to the HAuCl₄ is (1˜8):(0.15˜0.6).

4. The method for preparing Au—Ru nanoenzymes according to claim 3, wherein before adding the NaBH₄ aqueous solution to the mixture, the NaBH₄ aqueous solution needs to be pre-cooled, after pre-cooling treatment, a temperature of the NaBH₄ aqueous solution is 4° C.

5. Au—Ru nanoenzymes prepared by the method of preparing the Au—Ru nanoenzymes according to claim 1.

6. A method for preparing ATBMzyme nanoenzymes, the method comprising: dissolving the Au—Ru nanoenzymes according to claim 5 in water to obtain an Au—Ru nanoenzymes solution; adding 1-ethyl-3-(3-dimethylaminopropyl)carbodiimide and N-hydroxysuccinimide to the Au—Ru nanoenzymes solution, stirring and reacting the solution, and then adding brain natriuretic peptide to a reaction system, continuing to stir and react the reaction system; after the reaction is finished, collecting precipitate by centrifuging, and the precipitate is lyophilized to obtain the ATBMzyme nanoenzymes.

7. The method for preparing ATBMzyme nanoenzymes according to claim 6, wherein a mass ratio of the Au—Ru nanoenzymes to the 1-Ethyl-3-(3-dimethylaminopropyl)carbodiimide is (1˜4):(1˜20); a mass ratio of the Au—Ru nanoenzymes to the N-hydroxysuccinimide is (5˜20):(4˜15); a mass ratio of the brain natriuretic peptide to the Au—Ru nanoenzymes is (0.2˜1):(5˜20).

8. ATBMzyme nanoenzymes prepared by the method of preparing the ATBMzyme nanoenzymes according to claim 7.

9. An application of the ATBMzyme nanoenzymes according to claim 8 in preparing drugs for preventing, relieving or/and treating cardiotoxicity caused by a chemotherapy drug, wherein the chemotherapy drug is DOX.

10. A drug for preventing, relieving or/and treating cardiotoxicity caused by a chemotherapy drug, wherein the drug contains the ATBMzyme nanoenzymes according to claim 8.

11. The method for preparing Au—Ru nanoenzymes according to claim 2, wherein a molar ratio of the N-acetylcysteine to the HAuCl₄ is (1˜10):1, a molar ratio of the tannic acid to the HAuCl₄ is (1˜10):(1˜10), and a molar ratio of the NaBH₄ to the HAuCl₄ is (1˜8):(0.15˜0.6).

12. Au—Ru nanoenzymes prepared by the method of preparing the Au—Ru nanoenzymes according to claim 2.

13. Au—Ru nanoenzymes prepared by the method of preparing the Au—Ru nanoenzymes according to claim 3.

14. Au—Ru nanoenzymes prepared by the method of preparing the Au—Ru nanoenzymes according to claim 4.
